(19) United States
(12) Patent Application Publication
(10) Pub. No.: US 2020/0061350 A1
(43) Pub. Date: Feb. 27, 2020

(54) BALLOON WITHIN BALLOON CATHETER SYSTEM AND METHODS OF USE

(71) Applicant: Q3 MEDICAL DEVICES LIMITED, Dublin (IE)

(72) Inventor: Eric K. MANGIARDI, Charlotte, NC (US)

(21) Appl. No.: 16/113,251

(22) Filed: Aug. 27, 2018

(51) Int. Cl. A61M 25/10

(52) U.S. Cl. CPC A61M 25/1011; A61M 25/10182; A61M 25/10185; A61M 2210/12; A61M 2210/106; A61M 2210/1064; A61M 2025/0098; A61M 2210/1089; A61M 2210/1035; A61M 2210/0681; A61M 2025/105; A61M 2025/1013; A61M 2025/1015; A61M 2210/105

(57) ABSTRACT

A drug delivery system is described. The system has a first fluid delivery reservoir and a second fluid delivery reservoir. The first reservoir is separately controllable from the second reservoir. The first reservoir and second reservoir are attached to a manifold having a first solution channel in fluid communication with the first reservoir and a second solution channel in fluid communication with the second reservoir. The manifold is connected to a catheter having a first lumen in fluid communication with the first solution channel and a second lumen in fluid communication with the second solution channel. The catheter has a first balloon in fluid communication with the first lumen and a second balloon in fluid communication with the second lumen. The first balloon is located within the second balloon. The first balloon is not in fluid communication with the second balloon and the second balloon is perforated. A method for using the system and a kit containing the system are also described.

### FIELD

The present disclosure relates to medicament delivery devices. More specifically, the present disclosure relates to catheters for delivering a drug or active ingredient to a lumen.

### BACKGROUND

Sclerotherapy has been used in the treatment of blood vessel malformations, such as spider veins and varicose veins, for over 150 years. The method includes multiple injections through the skin of a dilute sclerosing solution into the vessel, causing the vessel to shrink and then dissolve, being naturally reabsorbed by the body. It is common practice for the patient to require at least two treatment sessions separated by several weeks in order to completely shrink and dissolve the treated vessel.

Foam sclerotherapy is a technique that involves injecting “foamed sclerosant drugs” within a blood vessel using a pair of syringes—one with sclerosant in it and one with gas. The sclerosant drugs (e.g., sodium tetradecyl sulfate (STS) or polidocanol) are mixed with air or a physiological gas, such as carbon dioxide, in a syringe or by using mechanical pumps. This increases the surface area of the drug. The foam sclerosant drug is more efficacious than the liquid one in causing sclerosis (i.e., thickening of the vessel wall and sealing off the blood flow), because it displaces the blood in the vessel, avoiding dilution of the drug and causing maximal sclerosant action.

Sclerotherapy can also be performed using ultrasound guidance, particularly in order to treat larger varicose veins. A map of the patient's varicose veins is created using ultrasound and these veins are injected under real-time monitoring of the injections, also using ultrasound. The sclerosant can be observed entering the vein, and further injections performed so that all the abnormal veins are treated. Follow-up ultrasound scans are used to confirm closure of the treated veins, and any residual varicose veins can be identified and treated.

Complications of Sclerotherapy may include venous thromboembolism, visual disturbances, allergic reaction, thrombophlebitis, skin necrosis, and hyperpigmentation or a red treatment area. Most complications occur due to an intense inflammatory reaction to the sclerotherapy agent in the area surrounding the injected vein. If the sclerosant is injected outside the vein, tissue necrosis and scarring can result. Skin necrosis, can be cosmetically devastating to the patient, and may take months to heal, and is more likely when higher concentrations of sclerosing solutions are used. Blanching of the skin often occurs when STS is injected into arterioles. Telangiectatic matting, or the development of tiny red vessels, is unpredictable and usually must be treated with repeat sclerotherapy or laser. In addition, there are systemic complications that may occur when the sclerosant travels through the veins to the heart, lung and brain.

Systemic complications can occur in a number of situations where a drug or other active ingredient is carried away from the target site by blood flow to other parts of the body. This can occur due to the vessel not being fully occluded during the application of the drug or active ingredient.

In order to minimize or eliminate complications associated with such a procedure, and to increase the efficiency of delivering a drug or active ingredient, the present disclosure provides a system and method for the application of a drug or active ingredient to a target vessel via a catheter having a distal balloon within a balloon structure that both occludes the vessel via inflation of the inner balloon and delivers a drug or active ingredient directly to the occluded site via perforations in the outer balloon. The present system and method provides superior results by allowing single-handled manipulation of the delivery mechanism, the ability to separately control inner balloon inflation and outer balloon delivery, and differently sized delivery syringes to account for different volume requirements for inflation and drug or active ingredient delivery. Additionally, the present system provides the advantage of allowing the direct application of the drug or active ingredient using a smaller volume or dose.

### SUMMARY

One aspect of the present disclosure is related to a drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir.

Another aspect of the present disclosure relates to a method for treating a condition at a target location in a body lumen of a subject in need thereof, the method comprising: establishing an entry portal into the body lumen, introducing a guide wire through the entry portal to the target location, directing the guide wire through the body lumen to the target location, feeding a catheter along the guide wire, through the entry portal and to the target location; wherein the catheter is part of a drug delivery system, the drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir; expelling a first physiologically acceptable solution from the first fluid delivery reservoir, thereby inflating the first balloon and occluding the body lumen at the target site; expelling a second physiologically acceptable solution from the second fluid delivery reservoir, thereby applying the second physiologically acceptable solution to the target site through the perforations in the second balloon, and wherein the second physiologically acceptable solution further comprises a drug or active ingredient.

Another aspect of the present invention comprises a kit for a drug delivery system, the kit comprising: a catheter comprising a first lumen and a second lumen; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and a manifold, the manifold comprising a first solution channel adapted at its proximal end to be in fluid communication with a first fluid delivery reservoir, and a second solution channel adapted at its proximal end to be in fluid communication with the second fluid delivery reservoir; and wherein the distal end of the manifold is adapted to connect to the proximal end of the catheter, such that the first lumen of the catheter is in fluid communication with the first solution channel of the manifold and the second lumen of the catheter is in fluid communication with the second solution channel of the manifold.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 is a longitudinal view of an embodiment of the outer balloon.

FIG. 2 is an end view of an embodiment of the outer balloon.

FIG. 3 is a longitudinal view of an embodiment of the outer balloon.

FIG. 4 is a longitudinal section of the dual balloon.

FIG. 5 is a detail view of an outer balloon showing an exemplary hole pattern.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” “lower,” “distal,” and “proximal” as well as derivatives thereof, should be construed to refer to the orientation as then described or as shown in the drawing figure under discussion. These relative terms are for convenience of description and normally are not intended to require a particular orientation. Terms concerning attachments, coupling and the like, such as “connected,” “mounted,” and “attached,” refer to a relationship wherein structures are secured or attached to one another either directly or indirectly through intervening structures, as well as both movable or rigid attachments or relationships, unless expressly described otherwise.

Unless otherwise noted, technical terms are used according to conventional usage. However, as used herein, the following definitions may be useful in aiding the skilled practitioner in understanding the invention. Such definitions shall be applied, unless a different definition is given in the claims or elsewhere in this specification.

As used herein, the terms “drug” and “active ingredient” are inclusive of any component of a composition that can be delivered through a catheter and is intended to furnish pharmacological activity or other direct effect in the diagnosis, cure, mitigation, treatment, or prevention of disease, or to affect the structure or any function of the body of humans or other animals. Drugs or active ingredients include, but are not limited to, inorganic molecules, organic molecules, small molecule pharmaceutical agents, steroids, chemotherapeutic agents, sclerosing agents, cauterizing agents, hormones, NSAIDs, analgesics, anesthetics, antipyretics, nucleic acids, peptides, proteins, fusion proteins, recombinant proteins, immunotherapeutic agents, and antibodies (including antigen-binding fragments, chimeric, humanized, fully human, antibody fusion proteins, anti-tumor, antibody-drug conjugates and antibody-toxin (such as, but not limited to, ricin A chain or diphtheria toxin A) conjugates). As used herein, a “composition” may further include pharmaceutically acceptable carriers or excipients.

In some embodiments, an active ingredient of the present disclosure includes beads. Beads of the present disclosure may include beads for blocking a lumen, including a blood vessel, beads infused or coated with a drug or other active ingredient, and radioactive beads. In some embodiments, beads are infused or coated with a therapeutic substance that is in an inactive form and known as “pre-activation beads.” In an exemplary method, a composition comprising pre-activation beads is delivered through the present device to a target site, such as a tumor or a blood vessel. A second composition comprising an agent that activates the therapeutic substance is delivered to the target site through the same device or in any other suitable manner, such as remote injection or through a second catheter. The second composition can be delivered before, at the same time as, or following the composition comprising pre-activation beads.

A device of the present application is well suited for targeted delivery of immunotherapeutic agents to treat a subject in need thereof for a tumor or neoplastic tissue that is benign, pre-malignant, or malignant. For example, the device can be guided into an artery supplying blood to a neoplasm and the inner balloon inflated to seal off blood flow to the neoplasm. An immunotherapeutic agent can then be expressed through the holes in the outer balloon and into contact with the neoplasm. Exemplary types of tumors or neoplasms that can be treated with the present device include, but are not limited to, adenoma, fibroma, hemangioma, lipoma, keratosis, dysplasia, metaplasia, carcinoma (including, but not limited to, stomach, prostate, pancreas, lung, liver, colon, or breast), sarcoma (including, but not limited to, cartilage, bones, fat, or nerves), blastoma. (including, but not limited to, medulloblastoma and glioblastoma, retinoblastoma, osteoblastoma, or neuroblastoma), germ cell tumor (including, but not limited to, ovarian, testicular, brain, abdominal or chest).

One aspect of the present disclosure is related to a drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir.

In some embodiments, the first solution channel is a non-valved solution channel. In some embodiments, the second solution channel is a non-valved solution channel. In some embodiments, both the first solution channel and the second solution channel are non-valved solution channels.

In some embodiments, the first fluid delivery reservoir and the second fluid delivery reservoir are syringes.

In other embodiments, the first volume capacity of the first fluid delivery reservoir and the second volume capacity of the second fluid delivery reservoir are different. In a further embodiment, the first volume capacity is greater than the second volume capacity. In another further embodiment, the second volume capacity is greater than the first volume capacity.

In other embodiments, the perforations in the second balloon are smallest at the proximal end of the balloon and increase in size incrementally towards the distal end of the second balloon.

In still other embodiments, the perforations in the second balloon are at a lower numerical density at the proximal end of the balloon and increase in numerical density incrementally towards the distal end of the second balloon.

In even other embodiments, the first balloon is longitudinally ribbed, creating longitudinal fluid channels on its outer surface within the second balloon. In a further embodiment, the longitudinal channels spiral around the first balloon.

In some embodiments, the catheter comprises a strain-relief section at its proximal end, where it adjoins to the manifold.

In some embodiments, the manifold and catheter comprise an additional contiguous lumen for the passage of a guide wire.

In other embodiments, the manifold further comprises a valved port for the connection of a third fluid delivery reservoir, wherein the valved port is in fluid communication with the second non-valved solution channel.

In some embodiments, the drug delivery system further comprises a housing that attaches to the manifold and holds the first fluid delivery reservoir and the second fluid delivery reservoir.

Another aspect of the present disclosure relates to a method for treating a condition at a target location in a body lumen of a subject in need thereof, the method comprising: establishing an entry portal into the body lumen, introducing a guide wire through the entry portal to the target location, directing the guide wire through the body lumen to the target location, feeding a catheter along the guide wire, through the entry portal and to the target location; wherein the catheter is part of a drug delivery system, the drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first non-valved solution channel in fluid communication with the first fluid delivery reservoir and a second non-valved solution channel in fluid communication with the second fluid delivery reservoir; wherein the distal end of the manifold is connected to the proximal end of a catheter comprising a first lumen in fluid communication with the first non-valved solution channel of the manifold and a second lumen in fluid communication with the second non-valved solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir; expelling a first physiologically acceptable solution from the first fluid delivery reservoir, thereby inflating the first balloon and occluding the body lumen at the target site; expelling a second physiologically acceptable solution from the second fluid delivery reservoir, thereby applying the second physiologically acceptable solution to the target site through the perforations in the second balloon, and wherein the second physiologically acceptable solution further comprises a drug or active ingredient.

In some embodiments, the body lumen is a blood vessel.

In other embodiments, the body lumen is a lymphatic vessel.

In still other embodiments, the body lumen is selected from the group consisting of the small bowel, ascending colon, transverse colon, descending colon, sigmoid colon, rectum, esophagus, bile duct, pancreatic duct, urethra, ureter, bronchus, bronchiole, and sinus.

In some embodiments, the condition is a spider vein.

In other embodiments, the condition is a varicose vein.

In still other embodiments, the condition is a cancer.

In some embodiments, the drug or active ingredient comprises a sclerosing agent. In some further embodiments, the sclerosing agent comprises sodium tetradecyl sulfate or polidocanol. In some still further embodiments, the sclerosing agent is applied to the target location as a foam.

In other embodiments, the drug or active ingredient comprises a chemotherapeutic agent.

Another aspect of the present invention comprises a kit for a drug delivery system, the kit comprising: a catheter comprising a first lumen and a second lumen; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and a manifold, the manifold comprising a first solution channel adapted at its proximal end to be in fluid communication with a first fluid delivery reservoir, and a second solution channel adapted at its proximal end to be in fluid communication with the second fluid delivery reservoir; and wherein the distal end of the manifold is adapted to connect to the proximal end of the catheter, such that the first lumen of the catheter is in fluid communication with the first solution channel of the manifold and the second lumen of the catheter is in fluid communication with the second solution channel of the manifold.

In some embodiments, the first solution channel is a non-valved solution channel. In some embodiments, the second solution channel is a non-valved solution channel. In some embodiments, both the first solution channel and the second solution channel are non-valved solution channels.

In some embodiments, the kit further comprises a first fluid delivery reservoir and a second fluid delivery reservoir.

In other embodiments, the kit further comprises a guide wire.

### Dual-Balloon Catheter

The catheter of the present disclosure comprises a proximal end and a distal end. The catheter comprises a dual-balloon structure at or proximal to its distal end, with an inner (first) balloon being fully enclosed within a perforated outer (second) balloon. The two balloons are connected to separate lumens within the catheter such that the fluid contents of the inner balloon and outer balloon are never in fluid communication with one another in the assembled drug delivery system.

In some embodiments, the balloons are composed of nylon 12. The balloons must be able to withstand a nominal pressure of 7 ATM. In some embodiments, the balloons must be able to withstand a burst pressure of 10-15 ATM. The balloon material should not contain a total area of embedded particulate material greater than 1.0 sq. mm, per TAPPI chart.

The outer balloon is perforated in order to allow a pharmaceutically acceptable carrier solution comprising a drug or active ingredient to be expelled from the outer balloon and contact the tissues at a target site. Depending on the viscosity of the solution comprising a drug or active ingredient and desired flow rate, in order to insure an even or uneven distribution of the drug or active ingredient being applied to the target site, the size of the perforations in the outer balloon increases from being smallest at the proximal end of the configuration to being largest at the distal end of the configuration. As a non-limiting example, the perforations at the proximal end are 0.5 mm in diameter and increase in size at a rate of 0.25 mm per linear centimeter towards the distal end of the outer balloon. In some embodiments, the most proximal perforations are about or equal to 0.1, 0.2, 0.3, 0.4, 0.5, 0.6, 0.7, 0.8, 0.9, or 1.0 mm in diameter. In some embodiments, the size of the perforations increases from proximal to distal at a rate of about or equal to 0.1, 0.2, 0.25, 0.3, 0.4, 0.5, 0.6, 0.7, 0.8, 0.9, or 1.0 mm per linear centimeter. Varying the size of the perforations, dependent upon the application, allows adjustment of the pressure gradients between the inner and outer balloons, thus allowing the solution to flow in equal distribution throughout the space between the inner balloon and the outer perforated balloon. In some applications, it may be desirable to have a greater rate of dispensing the solution towards the proximal end of the balloon, in which case the gradient is reversed, with the openings being largest at the proximal end.

The length of the dual balloon is dependent upon the size of the target site. In various embodiments, the dual balloon is about or equal to 0.5, 1, 2, 3, 4, 5, 6, 7, 8, 9, or 10 cm in length.

In some embodiments, the outer balloon is coated with, or comprises an embedded, drug or active ingredient. In some embodiments, the drug or active ingredient is different from the solution comprising a drug or active ingredient being delivered through the catheter. In other embodiments, the drug or active ingredient is the same as the solution comprising a drug or active ingredient being delivered through the catheter. In some embodiments, the drug or active ingredient coated on, or embedded in, the outer balloon is a sclerosing agent. In some further embodiments, the drug or active ingredient coated on, or embedded in, the outer balloon comprises sodium tetradecyl sulfate, while the solution comprising a drug or active ingredient being delivered through the catheter comprises polidocanol. In other further embodiments, the drug or active ingredient coated on, or embedded in, the outer balloon comprises polidocanol, while the solution comprising a drug or active ingredient being delivered through the catheter comprises sodium tetradecyl sulfate. In still other further embodiments, the drug or active ingredient coated on, or embedded in, the outer balloon comprises a chemotherapeutic agent, while the solution comprising a drug or active ingredient being delivered through the catheter comprises a sclerosing agent. In some embodiments, the drug or active ingredient coated on, or embedded in, the outer balloon is a therapeutic agent that is in an inactive form and the solution delivered through the catheter comprises an activating agent.

FIG. 1 depicts an exemplary embodiment of the outer balloon 100 designed to deliver a solution comprising a drug or active ingredient to a treatment area of interest, such as a varicose vein. In some embodiments, the perforated portion 101 of the outer balloon 1001 is between about 70 mm and about 110 mm in length. In other embodiments, the perforated portion 101 of the outer balloon 100 is between about 80 mm and about 100 mm in length. In particular embodiments, the perforated portion 101 of the outer balloon 100 is about 90 mm in length.

The pre- and post-inflation diameter of the balloon can be any size suitable for inflation within the target lumen to secure the balloon against the walls of the lumen. In some embodiments, the nominal (pre-inflation) diameter 102 of the outer balloon 100 is between about 1 mm and about 11 mm. In other embodiments, the nominal diameter 102 of the outer balloon 100 is between about 6 mm and about 10 mm. In still other embodiments, the nominal diameter 102 of the outer balloon 100 is between about 7 mm and about 9 mm. In particular embodiments, the nominal diameter 102 of the outer balloon 100 is about 8 mm.

The outer balloon 100 comprises a distal collar 103 and a proximal collar 104 for securing onto the catheter. Because the two longitudinal lumens for fluid delivery terminate in the dual balloons, the distal collar 103 is sized to be attached to, and form a distal seal on, the guide wire tube portion of the catheter, which extends through the balloons. In some embodiments, the distal collar 103 is between about 1 mm and about 3 mm in diameter. In other embodiments the distal collar 103 is about 2.09 mm in diameter. The proximal collar 104 is sized to be attached to, and form a proximal seal on, the catheter portion comprising the guide wire tube portion and the two longitudinal lumens for fluid delivery to the dual balloon. In some embodiments, the proximal collar 104 is between about 1 mm and about 4 mm in diameter. In other embodiments, the proximal collar 104 is between about 2 mm and about 3 mm in diameter. In particular embodiments the proximal collar 104 is about 2.6 mm in diameter.

In some embodiments, the length of the distal collar 103 is between about 1 mm and about 10 mm. In other embodiments, the length of the distal collar 103 is between about 3 mm and about 7 mm. In still other embodiments, the length of the distal collar 103 is between about 4 mm and about 6 mm. In particular embodiments, the length of the distal collar 103 is about 5.04 mm.

In some embodiments, the length of the proximal collar 104 is between about 1 mm and about 10 mm. In other embodiments, the length of the proximal collar 104 is between about 3 mm and about 8 mm. In still other embodiments, the length of the proximal collar 104 is between about 4 mm and about 7 mm. In particular embodiments, the length of the proximal collar 104 is about 5.66 mm.

The outer balloon 100 further comprises a distal shoulder 107 and a proximal shoulder 108 connecting the perforated portion 101 of the outer balloon 100 to the distal collar 103 and proximal collar 104, respectively. In some embodiments, the distal shoulder 107 has a linear length 109 of between about 3 mm and about 9 mm. In other embodiments, the distal shoulder 107 has a linear length 109 of between about 5 mm and about 7 mm. In particular embodiments, the distal shoulder 107 has a linear length 109 of about 6.06 mm. In some embodiments, the proximal shoulder 108 has a linear length 111 of between about 3 mm and about 10 mm. In other embodiments, the proximal shoulder 108 has a linear length 111 of between about 5 mm and about 8 mm. In particular embodiments, the proximal shoulder 108 has a linear length 111 of about 6.68 mm.

In some embodiments, the distal shoulder 107 connects from the distal collar 103 to the perforated portion 101 of the outer balloon 100 at an angle 110 between about 20 degrees and about 32 degrees. In other embodiments, the angle 110 of the distal shoulder 107 is between about 22 degrees and about 30 degrees. In still other embodiments, the angle 110 of the distal shoulder 107 is between about 24 degrees and about 28 degrees. In particular embodiments, the angle 110 of the distal shoulder 107 is about 26 degrees.

In some embodiments, the proximal shoulder 108 connects from the distal collar 104 to the perforated portion 101 of the outer balloon 100 at an angle 112 between about 16 degrees and about 28 degrees. In other embodiments, the angle 112 of the proximal shoulder 108 is between about 18 degrees and about 26 degrees. In still other embodiments, the angle 112 of the proximal shoulder 108 is between about 20 degrees and about 24 degrees. In particular embodiments, the angle 112 of the proximal shoulder 108 is about 22 degrees.

In some embodiments, the perforations 113 in the perforated portion 101 of the outer balloon 100 are arranged in rows encircling the balloon. In some embodiments, there are between 3 and 10 perforations 113 in each row. In other embodiments, there are between 4 and 9 perforations 113 in each row. In still other embodiments, there are between 5 and 8 perforations 113 in each row. In particular embodiments, there are 6 perforations 113 in each row. In some embodiments, the number of perforations 113 in a row increases moving from the proximal shoulder 108 towards the distal shoulder 107, thus increasing the density of perforations 113 towards the distal shoulder 107. In other embodiments, the number of perforations 113 in a row decreases moving from the proximal shoulder 108 towards the distal shoulder 107, thus decreasing the density of perforations 113 towards the distal shoulder 107.

In some embodiments, the rows of perforations 113 are staggered so that adjacent rows 114 of perforations are offset from one another. In some embodiments, every other row 115 of perforations is aligned so that the perforations lie along the same longitudinal line 116.

In some embodiments, there is between about 1 mm and about 7 mm of space between adjacent rows 114. In other embodiments, there is between about 2 mm and about 6 mm of space between adjacent rows 114. In still other embodiments, there is between about 3 mm and about 5 mm of space between adjacent rows 114. In particular embodiments, there is about 3.96 mm of space between adjacent rows 114. In some embodiments, adjacent rows 114 of perforations 113 get closer together moving from the proximal shoulder 108 towards the distal shoulder 107, thus increasing the density of perforations 113 towards the distal shoulder 107. In other embodiments, adjacent rows 114 of perforations 113 get farther apart moving from the proximal shoulder 108 towards the distal shoulder 107, thus increasing the density of perforations 113 towards the distal shoulder 107.

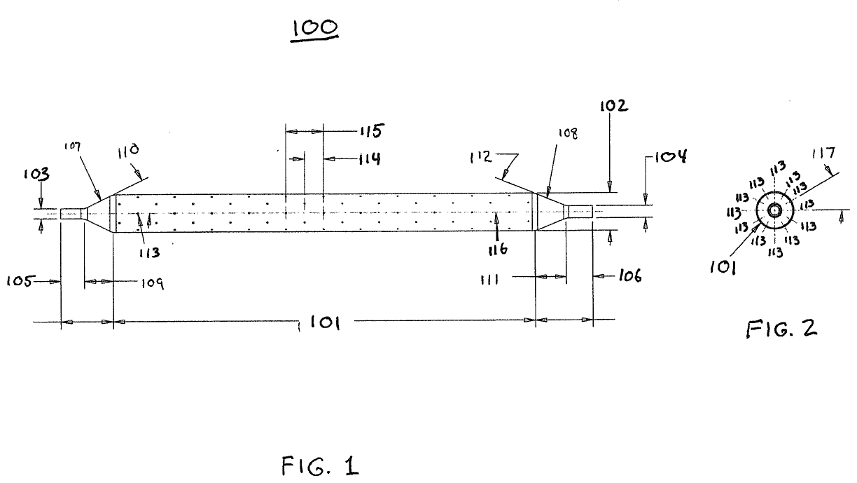

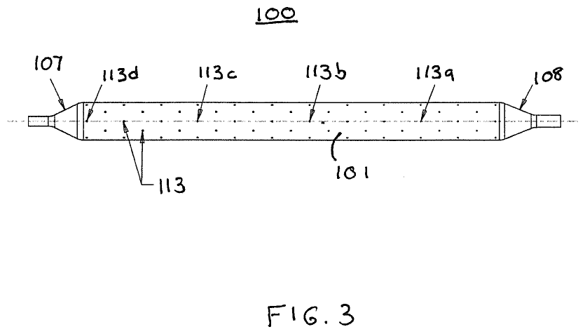

Turning now to FIG. 2, an end view of an exemplary outer balloon 100 is depicted. FIG. 2 is an exemplary embodiment wherein each row contains six perforations 113 encircling the perforated portion 101, resulting in an angle of 60 degrees between the perforations in a row. In this example, adjacent rows 114 (see FIG. 1) are staggered and every other row 115 of perforations is aligned so that the perforations lie along the same longitudinal line 116. This configuration results in perforations every 30 degrees 117 encircling the perforated portion 101.

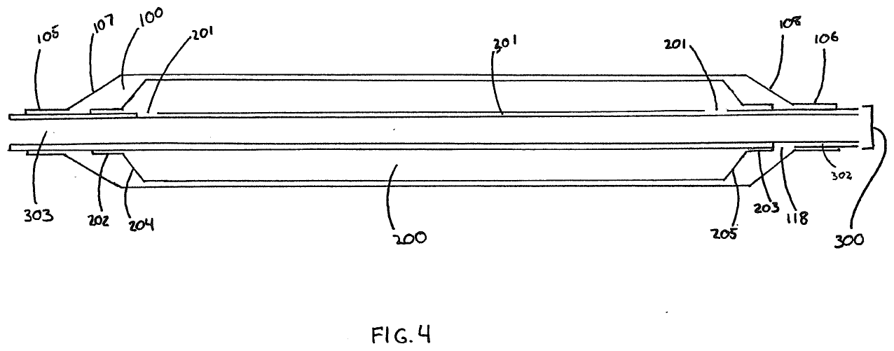

FIG. 3 shows an exemplary embodiment where the size of the perforations 113 in the perforated portion 101 of the outer balloon 100 increases moving from the proximal shoulder 108 towards the distal shoulder 107. For example, in some embodiments, a first row or group of rows of perforations 113a may be about 0.20 mm to about 0.26 mm in diameter. In other embodiments, a first row or group of rows of perforations 113a may be about 0.21 mm to about 0.25 mm in diameter. In still other embodiments, a first row or group of rows of perforations 113a may be about 0.22 mm to about 0.24 mm in diameter. In particular embodiments, a first row or group of rows of perforations 113a may be about 0.23 mm in diameter.

In some embodiments, a subsequent row or group of rows of perforations 113b may be about 0.25 mm to about 0.31 mm in diameter. In other embodiments, a subsequent row or group of rows of perforations 113b may be about 0.26 mm to about 0.30 mm in diameter. In still other embodiments, a subsequent row or group of rows of perforations 113b may be about 0.27 mm to about 0.29 mm in diameter. In particular embodiments, a subsequent row or group of rows of perforations 113b may be about 0.28 mm in diameter.

In some embodiments, another subsequent row or group of rows of perforations 113c may be present in the perforated portion 101 of the outer balloon 100. An exemplary another subsequent row or group of rows of perforations 113c may be about 0.30 mm to about 0.36 mm in diameter. In other embodiments, another subsequent row or group of rows of perforations 113c may be about 0.31 mm to about 0.35 mm in diameter. In still other embodiments, another subsequent row or group of rows of perforations 113c may be about 0.32 mm to about 0.34 mm in diameter. In particular embodiments, another subsequent row or group of rows of perforations 113c may be about 0.33 mm in diameter.

In some embodiments, yet another subsequent row or group of rows of perforations 113d may be present in the perforated portion 101 of the outer balloon 100. An exemplary yet another subsequent row or group of rows of perforations 113d may be about 0.35 mm to about 0.41 mm in diameter. In other embodiments, yet another subsequent row or group of rows of perforations 113d may be about 0.36 mm to about 0.40 mm in diameter. In still other embodiments, yet another subsequent row or group of rows of perforations 113d may be about 0.37 mm to about 0.39 mm in diameter. In particular embodiments, another subsequent row or group of rows of perforations 113d may be about 0.38 mm in diameter.

While the example depicted in FIG. 3 comprises four sets of rows of perforations 113 increasing in diameter from the proximal shoulder 108 towards the distal shoulder 107, it is understood that an outer balloon 100 of the present disclosure can comprise as few or many rows or sets of rows of perforations 113 of differing sizes as needed for a particular application. For example, a perforated portion 101 of the outer balloon 100 of the presently disclosed device may contain up to 2, 3, 4, 5, 6, 7, 8, 9, 10, 15, 20, 25, 30, or 40 rows or sets of rows of perforations 113 of differing sizes.

In some embodiments, the most proximal and most distal rows of perforations 113 are spaced a sufficient distance from the proximal shoulder 108 and the distal shoulder 107, respectively, to prevent leakage of a solution expressed through the perforations 113 past the ends of the balloon 100 onto non-target tissues or into bodily fluids. In some embodiments, the sufficient distance is at least about 1, 2, 3, 4, 5, 6, 7, 8, 9, 10, 12 or 15 mm from the respective shoulder.

In some embodiments, the outer balloon is perforated only near the distal end. In further embodiments, only the distal ½, ⅓, ¼, ⅕, or 1/10 portion of the balloon is perforated.

In other embodiments, the outer balloon is perforated only near the proximal end. In further embodiments, only the proximal ½, ⅓, ¼, ⅕, or 1/10 portion of the balloon is perforated.

In still other embodiments, the outer balloon is perforated only in its central region. In further embodiments, only the central ½, ⅓, ¼, ⅕, or 1/10 portion of the balloon is perforated.

In some embodiments, the outer balloon may comprise a bulbous collar or ring before, on, or after the proximal shoulder 108 or the distal shoulder 107 in order to contain a solution expressed through the perforations 113 to prevent or reduce seepage of the solution past the ends of the balloon 100 onto non-target tissues or into bodily fluids.

FIG. 4 represents an exemplary structural configuration of an exemplary longitudinal section through the outer balloon 100, inner balloon 200 and the catheter 300.

The inner balloon comprises an impermeable surface. The balloon is inflatable with a pharmaceutically acceptable solution, such as sterile normal saline solution. Inflation of the inner balloon serves two purposes in the function of the dual-balloon. The first is the occlusion of the lumen at the target site, so that the flow of bodily fluids, such as blood or lymph fluid, is stopped at the target site during treatment. This allows the use of a lower amount of drug or active ingredient to be effectively used in the treatment, as well as reduces the likelihood of complications due to the drug or active ingredient being carried to other parts of the body by the bodily fluid.

The second purpose of the inner balloon is to press the outer balloon into direct contact with the target site in the bodily lumen. As a solution comprising a drug or active ingredient is expressed into the outer balloon from the catheter, the pressure from the inner balloon forces the solution through perforations in the outer balloon. The pressure exerted by the inner balloon then forces the solution to spread across the surface of the outer balloon, coating the walls of the lumen at the target site.

In some embodiments, the surface of the inner balloon is ribbed, forming channels between the balloons and directing the solution in the outer balloon containing the drug or active ingredient. The ribs may form linear, spiral or undulating channels, dependent upon the application that the system is being used for.

The inner balloon 200 comprises a distal collar 202 and a proximal collar 203 for securing onto the catheter. The inner balloon 200 further comprises a distal shoulder 204 and a proximal shoulder 205 connecting the walls of the inner balloon 200 to the distal collar 202 and proximal collar 203, respectively.

The catheter 300 comprises two longitudinal lumens for fluid delivery to the dual balloon. A first lumen 301 of the catheter 300 is in fluid communication with the inner balloon 200 via at least one port 201 and delivers a physiologically suitable solution for inflation of the inner balloon 200. In some embodiments, the port 201 is located at or near the distal end of the inner balloon 200. In other embodiments, the port 201 is located at or near the proximal end of the inner balloon 200. In still other embodiments, there is a port 201 located at or near each end of the inner balloon 200. In other embodiments, there are multiple ports 201 located along the length of the inner balloon 200.

A second lumen 302 of the catheter 300 is in fluid communication with the outer balloon 100 via at least one port 118 and delivers a physiologically suitable solution comprising at least one drug or active ingredient to the outer balloon 100 for exposure to tissue at the target site.

In some embodiments, the catheter 300 further comprises a lumen 303 for the passage of a guide wire. In other embodiments, the catheter comprises a stress relief region at its proximal end. In some embodiments, the flexibility of the catheter varies over its length, with the catheter being most flexible towards its distal tip and stiffer towards the proximal end in order to enhance pushability of the catheter.

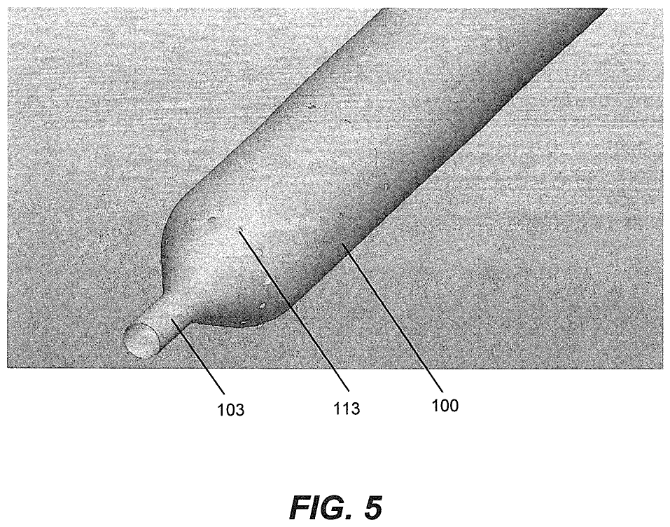

FIG. 5 shows the distal end of an exemplary outer balloon 100, showing an exemplary hole 133 pattern and the distal collar 103. The holes 113 can be of any shape that is suitable for the fluid or beads being expressed through the holes. In addition to the pattern of the holes in the outer balloon 100, in some embodiments, the shape of the holes allows for better control of flow or distribution of the fluid or beads being expressed through the holes. In some embodiments, the holes 113 are round. In other embodiments, the holes 113 can be oblong, oval, elliptical, arc-shaped, crescent-shaped, semi-circular, or polygonal in shape.

### Manifold

Another element of the present disclosure relates to a manifold for attaching the catheter to at least two fluid delivery reservoirs. The manifold comprises a first solution channel adapted at its proximal end to be in fluid communication with a first fluid delivery reservoir, and a second solution channel adapted at its proximal end to be in fluid communication with a second fluid delivery reservoir. The distal end of the manifold is adapted to connect to the proximal end of the catheter, such that the first lumen of the catheter is in fluid communication with the first solution channel of the manifold and the second lumen of the catheter is in fluid communication with the second solution channel of the manifold.

In some embodiments, the manifold comprises an additional fluid inlet for attaching a third fluid delivery reservoir. This additional fluid inlet is in fluid communication with the second solution channel and may be valved or have a cap or plug to prevent fluid egress when not in use. The additional fluid inlet allows the introduction of at least one additional solution into the outer balloon. The additional solution may be a flushing solution, such as normal saline, or it may comprise at least one drug or active ingredient to be applied before, during or after application of the drug or active ingredient from the second fluid delivery reservoir.

In some embodiments, the first solution channel is a non-valved solution channel. In some embodiments, the second solution channel is a non-valved solution channel. In some embodiments, both the first solution channel and the second solution channel are non-valved solution channels.

### Reservoirs

The drug delivery system of the present disclosure comprises a pair of independent fluid delivery reservoirs that attach to separate ports on the proximal end of the manifold. The fluid delivery reservoirs can take any suitable for fluid retention and evacuation known in the art. In some embodiments, a fluid delivery reservoir of the present disclosure is a syringe. In other embodiments, a fluid delivery reservoir of the present system is a bladder. Fluid delivery reservoirs of the present system may be operated by any suitable means including, but not limited to, manual operation, machine control, computer control or robotic control.

In some embodiments, the fluid delivery reservoirs may be operated by a syringe pump. In some embodiments, the operation of the syringe pump is programmable. In other further embodiments, each fluid delivery reservoir is operated by a separate syringe pump having an operation speed independent of other syringe pumps. In still other further embodiments, the fluid delivery reservoirs may be operated by a single or integrated syringe pump, wherein the syringe pump can operate each fluid delivery reservoir at a speed independent of the operation of other fluid delivery reservoirs.

The first fluid delivery reservoir for inflating the inner balloon of the catheter is attachable to a first proximal port for the first solution channel of the manifold. The attachment can be made by any suitable means known in the art including, but not limited to threaded, luer slip or luer lock. The size of the first fluid delivery reservoir can be dependent upon the length of the inner balloon and the inner diameter of the lumen at the target site. In some embodiments, the volume of the first fluid delivery reservoir equals or is about 1, 3, 5, 10 or 15 ml. In some embodiment, the first solution channel is a non-valved solution channel.

A second fluid delivery reservoir for supplying a solution containing a drug or active ingredient to the outer balloon of the catheter is attachable to a second proximal port for the second solution channel of the manifold. The attachment can be made by any suitable means known in the art including, but not limited to threaded, luer slip or luer lock. The size of the second fluid delivery reservoir can be dependent upon the length of the outer balloon, dosage requirements, and the surface area of the lumen at the target site. In some embodiments, the volume of the second fluid delivery reservoir equals or is about 1, 3, 5, 10 or 15 ml. In some embodiment, the second solution channel is a non-valved solution channel.

In some embodiments, the volume, length or diameter of the first fluid delivery reservoir is larger than the second fluid delivery reservoir. This may be the case when inner balloon is of a great length, or the inner diameter of the lumen at the target site is large, but the amount of drug- or active ingredient-containing solution needed to effectively treat the target site is small.

In other embodiments, the volume, length or diameter of the first fluid delivery reservoir is smaller than the second fluid delivery reservoir. This may be the case when inner balloon is of a short length, or the inner diameter of the lumen at the target site is narrow, but the amount of drug- or active ingredient-containing solution needed to effectively treat the target site is great.

In still other embodiments, the first fluid delivery reservoir is the same size as the second fluid delivery reservoir.

In some embodiments, the system further comprises at least a third, or additional, fluid reservoir. In some embodiments, the additional fluid reservoir connects to an additional fluid inlet of the manifold, which is in fluid communication with the second solution channel. In other embodiments, the additional fluid reservoir connects to the second proximal port of the manifold in place of the second fluid delivery reservoir.

The size of an additional fluid reservoir is independent of the size of the first and second fluid reservoirs, being dependent instead upon the nature/purpose of the solution it contains. In some embodiments, an additional fluid reservoir contains a flush or wash solution that is dispensed to the target site through the perforations in the outer balloon before or after dispensing the drug or active ingredient from the second fluid delivery reservoir. In other embodiments, an additional fluid reservoir contains an additional drug or active ingredient for application to the target site through the perforations in the outer balloon before, after, or simultaneously with the drug or active ingredient solution dispensed from the second fluid delivery reservoir.

In some embodiments, the first fluid delivery reservoir and the second fluid delivery reservoir are separately controllable. The inner balloon is inflated with a physiologically acceptable solution from the first fluid delivery reservoir, such as sterile normal saline, thereby pressing the outer balloon against the walls of the body lumen at the target site. Solution containing a drug or active ingredient is then dispensed from the second fluid delivery reservoir into the compressed space between the inner and outer balloons and forced through the perforations in the outer balloon into contact with the target tissue.

In other embodiments, the first fluid delivery reservoir and the second fluid delivery reservoir are linked and controllable together. In some further embodiments, the linked first fluid delivery reservoir and second fluid delivery reservoir are of different volumes or diameters such that, when actuated, the first delivery reservoir delivers fluid to the inner balloon at a rate that is greater than the delivery of fluid from the second delivery reservoir to the outer balloon, or vice versa, dependent upon the application.

In some embodiments, at least the first fluid delivery reservoir and the second fluid delivery reservoir are contained within a common holder. The holder is capable of containing reservoirs that are of the same or different volumes, lengths or diameters.

### Method

The system of the present disclosure provides a device for performing a method for treating a condition at a target location in a body lumen of a subject in need thereof. The method comprises the steps of establishing an entry portal into the body lumen, introducing a guide wire through the entry portal to the target location, directing the guide wire through the body lumen to the target location, feeding a catheter along the guide wire, through the entry portal and to the target location; wherein the catheter is part of a drug delivery system, the drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir; delivering a first physiologically acceptable solution from the first fluid delivery reservoir, thereby inflating the first balloon and occluding the body lumen at the target site; delivering a second physiologically acceptable solution from the second fluid delivery reservoir, thereby applying the second physiologically acceptable solution to the target site through the perforations in the second balloon, and wherein the second physiologically acceptable solution further comprises a drug or active ingredient.

In some embodiments, the method further comprises applying at least one additional physiologically acceptable solution to the target site through the perforations in the second balloon. In some embodiments, the at least one additional physiologically acceptable solution further comprises a drug or active ingredient. In other embodiments, the at least one additional physiologically acceptable solution is an activator for the first drug or active ingredient. In other embodiments, the at least one additional physiologically acceptable solution is a flushing or rinsing solution that does not contain a drug or active ingredient.

In some embodiments, the first solution channel is a non-valved solution channel. In some embodiments, the second solution channel is a non-valved solution channel. In some embodiments, both the first solution channel and the second solution channel are non-valved solution channels.

In some embodiments, the establishment of an entry portal comprises making an incision through a tissue of the body, such as the skin or the wall of a vessel. In other embodiment, the establishment of an entry portal comprises inserting the guide wire or catheter through a natural body opening, such as the mouth, nares, anus, or urethra.

The method of the present disclosure can be applied to a variety of body lumens as target sites.

In some embodiments, the body lumen is a blood or lymphatic vessel. In some further embodiments, the blood vessel is an artery. In some embodiments, the artery is an artery of the head, neck, thorax, vertebral column, abdomen, pelvis, arm, hand, leg or foot. In some still further embodiments, the artery can be, but is not limited to, a systemic artery, pulmonary artery, aorta, subclavian artery, carotid artery, right coronary artery, right marginal artery, posterior descending artery, left coronary artery, circumflex artery, left anterior descending artery, axillary artery, brachial artery, radial artery, ulnar artery, iliac artery, femoral artery, popliteal artery, tibial artery, dorsalis pedis artery, arch of foot artery, superior mesenteric artery, descending aorta, thoracic aorta, abdominal aorta, or renal artery.

In other further embodiments, the blood vessel is a vein. In some embodiments, the vein is a vein of the head, neck, thorax, vertebral column, abdomen, pelvis, arm, hand, leg or foot. In some embodiments, the vein is a superficial vein, deep vein, perforator vein, communicating vein, or systemic vein. In some further embodiments, the vein can be, but is not limited to, a jugular vein, subclavian vein, axillary vein, pulmonary vein, cephalic vein, superior vena cava, inferior vena cava, mesenteric vein, basilic vein, renal vein, femoral vein, saphenous vein or coronary vein. In other further embodiments, the vein is a spider vein. In yet other further embodiments, the vein is a varicose vein.

In some embodiments, a blood vessel treatable with the present device is between about 1 mm and about 30 mm in diameter. In some embodiments, the diameter of the blood vessel is between about 1 mm and about 5 mm, between about 1 mm and about 10 mm, between about 1 mm and about 15 mm, between about 1 mm and about 20 mm, between about 1 mm and about 25 mm, between about 5 mm and about 10 mm, between about 5 mm and about 15 mm, between about 5 mm and about 20 mm, between about 5 mm and about 25 mm, between about 10 mm and about 15 mm, between about 10 mm and about 20 mm, between about 10 mm and about 25 mm, between about 10 mm and about 30 mm, between about 15 mm and about 20 mm, between about 15 mm and about 25 mm, between about 15 mm and about 30 mm, between about 20 mm and about 25 mm, between about 20 mm and about 30 mm, between about 25 mm and about 30 mm, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mm.

In other embodiments, the body lumen is the larynx, trachea, or bronchial tube. In some further embodiments, the bronchial tube is a mainstream bronchus, segmental bronchus or subsegmental bronchus.

In still other embodiments, the body lumen is the esophagus. In even other embodiments, the body lumen is the stomach, duodenum, small intestine, colon or rectum.

In yet other embodiments, the body lumen is a duct, such as the bile duct, or pancreatic duct.

In some embodiments, the body lumen is the urethra, urinary bladder, ureter, renal pelvis, or calyx.

In still other embodiments, the body lumen is a medullary cavity of a bone.

The method of the present disclosure can be used to deliver a variety of drugs or active ingredients body lumens as target sites.

In some embodiments, the drug or active ingredient is a sclerosing agent for the treatment of spider veins or varicose veins. In some further embodiments, the sclerosing agent is sodium tetradecyl sulfate or polidocanol.

In other embodiments, the drug or active ingredient is a chemotherapeutic agent for the treatment of a cancer.

In some embodiments, the drug or active ingredient is delivered to the target site under high pressure. For example, high pressure delivery may be useful for increasing the amount of the drug that is pushed into the tissues of the target site.

### Kit

A kit of the present disclosure contains a catheter. The catheter has a first lumen and a second lumen and, in the proximity of its distal end, a first balloon in fluid communication with the first lumen and a second balloon in fluid communication with the second lumen. The first balloon is located within the second balloon, wherein the first balloon is not in fluid communication with the second balloon, and wherein the second balloon is perforated.

In some embodiments, the kit contains a manifold for attaching the catheter to at least two fluid delivery reservoirs. The manifold comprises a first solution channel adapted at its proximal end to be in fluid communication with a first fluid delivery reservoir, and a second solution channel adapted at its proximal end to be in fluid communication with a second fluid delivery reservoir. The distal end of the manifold is adapted to connect to the proximal end of the catheter, such that the first lumen of the catheter is in fluid communication with the first solution channel of the manifold and the second lumen of the catheter is in fluid communication with the second solution channel of the manifold.

In some embodiments, the first solution channel is a non-valved solution channel. In some embodiments, the second solution channel is a non-valved solution channel. In some embodiments, both the first solution channel and the second solution channel are non-valved solution channels.

In some embodiments, the kit contains a guidewire for directing the introduction of the catheter from an entry portal to the target site. The catheter and manifold may be adapted to include an additional contiguous lumen for the passage of the guidewire.

In some embodiments, the kit contains a first fluid delivery reservoir and a second fluid delivery reservoir. In some embodiments, the fluid delivery reservoirs are syringes.

### Example 1: Treatment of Varicose Vein

A patient presents with varicosity of the small saphenous vein in the lower left leg, located in the superior ⅓ of the calf (gastrocnemius). The small saphenous vein is punctured distal (upstream) to the varicosity using the Seldinger technique performed under 1% lidocaine anesthesia. A guide wire is inserted through the incision and passed through the vein into the target region of the varicosity. The catheter and manifold segments of the presently described drug delivery system are passed onto the guide wire and the dual balloon is advanced to the target site. The guide wire is removed from the apparatus through the manifold.

Sterile saline solution is dispensed from the first fluid delivery reservoir until the balloon is inflated against the walls of the lumen. A 3% solution of sodium tetradecyl sulfate in sterile normal saline is dispensed from the second fluid delivery reservoir into the space between the inner and outer balloons. As the solution through the space, it is forced through the perforations in the outer balloon and into contact with the target tissue.

Following an effective amount of exposure of the target site to the drug or active ingredient, the sterile saline solution is withdrawn from the inner balloon into the first fluid delivery reservoir, deflating the balloon. The catheter is then withdrawn through the entry portal and the entry portal is closed.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A drug delivery system, comprising: a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir.

2. The drug delivery system of claim 1, wherein the first fluid delivery reservoir and the second fluid delivery reservoir are syringes.

3. The drug delivery system of claim 1, wherein the first volume capacity of the first fluid delivery reservoir and the second volume capacity of the second fluid delivery reservoir are different.

4. The drug delivery system of claim 1, wherein the perforations in the second balloon are smallest at the proximal end of the balloon and increase in size incrementally towards the distal end of the second balloon.

5. The drug delivery system of claim 1, wherein the perforations in the second balloon are at a lower numerical density at the proximal end of the balloon and increase in numerical density incrementally towards the distal end of the second balloon.

6. The drug delivery system of claim 1, wherein the catheter comprises a strain-relief section at its proximal end, where it adjoins to the manifold.

7. The drug delivery system of claim 1, wherein the manifold and catheter comprise an additional contiguous lumen for the passage of a guide wire.

8. The drug delivery system of claim 1, wherein the manifold further comprises a valved port for the connection of a third fluid delivery reservoir, wherein the valved port is in fluid communication with the second solution channel.

9. The drug delivery system of claim 1, wherein the drug delivery system further comprises a housing that attaches to the manifold and holds the first fluid delivery reservoir and the second fluid delivery reservoir.

10. The drug delivery system of claim 1, wherein the second balloon is coated with, or comprises an embedded, drug or active ingredient.

11. A method for treating a condition at a target location in a body lumen of a subject in need thereof, the method comprising:
establishing an entry portal into the body lumen, introducing a guide wire through the entry portal to the target location,
directing the guide wire through the body lumen to the target location,
feeding a catheter along the guide wire, through the entry portal and to the target location; wherein the catheter is part of a drug delivery system, the drug delivery system comprising a first fluid delivery reservoir of a first volume capacity fluid delivery and a second fluid delivery reservoir of a second volume capacity; wherein said first reservoir and said second reservoir are attached to the proximal end of a manifold having a first solution channel in fluid communication with the first fluid delivery reservoir and a second solution channel in fluid communication with the second fluid delivery reservoir; wherein the distal end of the manifold is connected to the proximal end of a catheter comprising a first lumen in fluid communication with the first solution channel of the manifold and a second lumen in fluid communication with the second solution channel of the manifold; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and wherein the first fluid delivery reservoir is separately controllable from the second fluid delivery reservoir;
expelling a first physiologically acceptable solution from the first fluid delivery reservoir, thereby inflating the first balloon and occluding the body lumen at the target site;
expelling a second physiologically acceptable solution from the second fluid delivery reservoir, thereby applying the second physiologically acceptable solution to the target site through the perforations in the second balloon, and wherein the second physiologically acceptable solution further comprises a drug or active ingredient.

12. The method of claim 11, wherein the body lumen is a blood vessel.

13. The method of claim 11, wherein the body lumen is a lymphatic vessel.

14. The method of claim 11, wherein the body lumen is selected from the group consisting of the small intestine, large intestine, esophagus, bile duct, pancreatic duct, urethra, ureter, bronchus, bronchiole, and sinus.

15. The method of claim 11, wherein the condition is a varicose vein or spider vein.

16. The method of claim 11, wherein the condition is a cancer.

17. The method of claim 11, wherein the drug or active ingredient comprises a sclerosing agent.

18. The method of claim 17, wherein the sclerosing agent comprises sodium tetradecyl sulfate or polidocanol.

19. The method of claim 17, wherein sclerosing agent is applied to the target location as a foam.

20. The method of claim 11, wherein the drug or active ingredient comprises a chemotherapeutic agent.

21. A kit for a drug delivery system, the kit comprising:
a catheter comprising a first lumen and a second lumen; the catheter further comprising in the proximity of its distal end a first balloon in fluid communication with the first lumen and a second balloon in fluid communication with the second lumen; wherein the first balloon is located within the second balloon, wherein the first balloon is not in fluid communication with the second balloon, and wherein the second balloon is perforated; and
a manifold, the manifold comprising a first solution channel adapted at its proximal end to be in fluid communication with a first fluid delivery reservoir, and a second solution channel adapted at its proximal end to be in fluid communication with the second fluid delivery reservoir; and wherein the distal end of the manifold is adapted to connect to the proximal end of the catheter, such that the first lumen of the catheter is in fluid communication with the first non-valved solution channel of the manifold and the second lumen of the catheter is in fluid communication with the second non-valved solution channel of the manifold.

22. The kit of claim 21, wherein the kit further comprises a first fluid delivery reservoir and a second fluid delivery reservoir.

23. The kit of claim 21, wherein the kit further comprises a guide wire.
